(19) United States
(12) Patent Application Publication
(10) Pub. No.: US 2020/0172841 A1
(43) Pub. Date: Jun. 4, 2020

(54) ALCOHOL-FREE WINE BEVERAGE

(71) Applicants: Donald F. Wilkes, Santa Monica, CA (US); Kelly Chavarrio, Montebello, CA (US)

(72) Inventors: Donald F. Wilkes, Santa Monica, CA (US); Kelly Chavarrio, Montebello, CA (US)

(21) Appl. No.: 16/209,941

(22) Filed: Dec. 4, 2018

(51) Int. Cl. C12G 1/00; C12G 3/08; A61K 9/00; A61K 31/05; A61K 9/107; A61K 31/352; A61K 31/522

(52) U.S. Cl. CPC C12G 1/00; C12G 3/08; A61K 9/0095; C12G 2200/21; A61K 9/1075; A61K 31/352; A61K 31/522; A61K 31/05

(57) ABSTRACT

A non-alcoholic wine beverage is disclosed. The non-alcoholic wine beverage may include a wine beverage that may have its alcohol content removed. The alcohol content may then be replaced with other ingredients that may provide effects similar to the desirable effects of alcohol while avoiding those effects that may be undesirable. For example, the non-alcoholic wine beverage may include one or more cannabinoids such as CBD and THC. The beverage may also include caffeine and other ingredients.

### COPYRIGHT STATEMENT

This patent document contains material subject to copyright protection. The copyright owner has no objection to the reproduction of this patent document or any related materials in the files of the United States Patent and Trademark Office, but otherwise reserves all copyrights whatsoever.

### FIELD OF THE INVENTION

This invention relates to beverages, and in particular, to alcohol-free wine beverages.

### BACKGROUND OF THE INVENTION

Wine has been consumed throughout the world for centuries. And as is widely known, wine may contain alcohol which may produce both desirable and undesirable effects on the consumer.

For example, the consumption of alcohol may cause temporary euphoria. As is known in the art, alcohol when ingested may increase the release of dopamine in the reward center (e.g., in the ventral striatum) of the consumer's brain. Dopamine functions as a neurotransmitter (a chemical released by neurons) that passes signals from one nerve cell to another in the brain along distinct dopamine pathways. One such dopamine pathway, the mesolimbic pathway, increases dopamine release in the nucleus accumbens resulting in the feeling of increased pleasure, joy, desire and motivation. In this way, the drinking of alcohol directly results in a feeling of euphoria and happiness.

In addition, the consumption of alcohol may also relieve symptoms of anxiety by depressing the central nervous system. However, while alcohol may provide temporary relief from anxiety, after its euphoric effects may wear off it may unfortunately cause an increase of anxiety, irritability and even depression. This may also lead to alcohol abuse disorders.

It is also known that alcohol acts as a pain killer by blocking N-methyl-D-aspartate receptors (also known as the NMDA receptors or NMDAR) in the central nervous system. And in fact, alcohol has historically been the main predecessor to modern anesthetics.

However, there are many negative effects of ingesting alcohol. Alcohol is known to increase the risk of heart disease, liver disease, cancer, and other diseases. Alcohol also causes shrinkage in the hippocampus, the brain area associated with memory and reasoning, and is linked to an increased risk of dementia and Alzheimer's disease.

Alcohol is also highly addictive and may lead to depression and other mental and/or mood disorders. Over time, with more drinking, the dopamine effect may diminish until it's almost nonexistent. However, at this stage, the consumer may often experience a strong compulsive need to continue drinking, often in excess, resulting in addiction and alcohol abuse disorders.

According to results from the 2001-2002 National Epidemiologic Survey on Alcohol and Related Conditions (NESARC), about 10 percent of men and 3 to 5 percent of women become alcoholics over the course of their lifetime.

Accordingly, there is a need for a wine beverage that may have its alcohol content removed and replaced with other ingredients that may provide effects similar to the desirable effects of alcohol while avoiding those effects that may be undesirable. There is also a need for a wine beverage that may include ingredients that may provide the desirable effects similar to alcohol but that may not be addictive.

### DETAILED DESCRIPTION OF THE PREFERRED EMBODIMENTS

In general, the product according to exemplary embodiments hereof includes an innovative non-alcoholic beverage. The beverage may be a wine beverage that may have at least a portion of its alcohol removed. The beverage may be a dealcoholized wine, a non-alcoholic wine, an alcohol-free wine or simply a wine beverage with no alcohol content. It is understood that the terms “alcohol-free” and “nonalcoholic” may be used interchangeably in this specification, and as used herein may be a wine derived beverage having less than 0.5% alcohol content considered by the Bureau of Alcohol, Tobacco Products and Firearms Division of the U.S. Treasury Department as not being a wine for tax purposes. It is also understood that if this limit varies at any time then the term “alcohol-free” as used herein may also vary accordingly.

As stated above, it is known in the art that alcohol when consumed may produce both desirable effects and undesirable effects. The desirable effects may include the relief of pain, the lessening of anxiety and the temporary feeling of euphoria and happiness. The undesirable effects may include an increased risk of heart disease, liver disease, cancer, and other diseases. Alcohol also causes shrinkage in the hippocampus, the brain area associated with memory and reasoning, and is linked to an increased risk of dementia and Alzheimer's disease. Alcohol is also highly addictive and may lead to depression.

According to exemplary embodiments described herein, the alcohol within the beverage may be removed and replaced with other ingredients that may produce effects similar to the desirable effects of alcohol, but without the adverse effects associated with alcohol consumption. In some exemplary embodiments, the added ingredients may also provide health benefits when consumed as will be described in later sections.

In some embodiments hereof, the beverage may include as a base, among other ingredients, the non-alcoholic wine beverage described in U.S. Pat. No. 9,445,623 filed on Apr. 30, 2009, the entire contents of which are hereby fully incorporated herein by reference for all purposes.

For example, one exemplary embodiment hereof of the wine beverage may include a dealcoholized red wine base, for example, including but not limited to, a wine base prepared from Cabernet Sauvignon, Merlot, Pinot Noir, Claret, Sangiovese or other red wine varietals. The beverage may be carbonated or non-carbonated. The beverage may also be blended and/or flushed with nitrogen.

In another exemplary embodiment, the wine beverage may include a dealcoholized white wine base, for example, including but not limited to, a wine base prepared from Sauvignon Blanc, Chardonnay or other white wine varietals.

It is understood by a person of ordinary skill in the art that the wine beverage may include a wine base that may be prepared from any wine varietals, from any combination of wine varietals, or from other types of bases and that the scope of the wine beverage is not limited in any way by the type or combinations of types of wine varietals that may be used.

The wine base may be dealcoholized, for example by the use of a Spinning Cone Column, a membrane reverse osmosis process or any other techniques or combinations thereof. It may be preferable that the resulting dealcoholized wine beverage not contain more than about 0.5% alcohol.

Once the alcohol has been removed from the wine, it may be replaced with ingredients specifically processed for the beverage. The innovative ingredients may be provided in specific proportions to provide benefits to the beverage and to the consumer of the beverage.

The non-alcoholic wine beverage will now be described in detail with respect to the various innovative ingredients that the beverage may include.

Cannabinoids

In one exemplary embodiment hereof, the non-alcoholic wine beverage may include one or more cannabinoids, individually or in any combination. Cannabinoid is a class of chemical compounds found in plants in the Cannabis family (e.g., hemp, marijuana, etc.). To date, at least 113 cannabinoids have been identified, accounting for up to 40% of the plant's extract. Well known cannabinoids include Cannabidiol (CBD), Tetrahydrocannabinol (THC) among others, each of which may include a variety of health benefits. In addition, cannabinoids are not addictive.

As will be described below, cannabinoids may be provided as cannabinoid isolates, full-spectrum cannabinoid oils, as other forms and in any combination thereof.

Cannabidiol (CBD)

In one exemplary embodiment hereof, the non-alcoholic wine beverage may include Cannabidiol (CBD). Cannabidiol (CBD) is one such naturally occurring cannabinoid constituent of Cannabis. While CBD may have no psychotropic effects when consumed, research has shown that CBD may act to relieve pain, to reduce inflammation (e.g., anti-inflammatory), to reduce anxiety (anxiolytic), reduce depression, help with insomnia, reduce post-traumatic stress disorder (PTSD) as well as provide other health benefits. CBD has also been shown to successfully treat multiple sclerosis, chronic pain, and epilepsy, as well as other ailments.

There are different varieties of the Cannabis species, including hemp marijuana, and others, all of which contain CBD.

CBD may be extracted from the plant using a variety of extraction techniques. In one example, the extraction process may be a solvent process that may use ethanol (or other type of solvent) to separate the plant's components. In a second example, a CO₂ extraction method may be used to pull the botanical CBD-rich oil from the plant. In this example, non-toxic supercritical CO₂ may be pushed through the plant material in a series of filtration chambers to extract the CBD oil from the plant. In any event, the resulting CBD oil may be a CBD-rich concentrate that may include all of the cannabinoids, fatty acids and other natural plant material from the Cannabis (marijuana or hemp).

The extraction techniques may yield CBD in a generally oil-based form. In this form, the CBD may be fat-soluble and hydrophobic. As such, while the CBD oil may mix well with oil based liquids, it may not mix well with other types of liquids (e.g., water based liquids).

Accordingly, it may be preferable to further process or otherwise affect the CBD oil such that it may be mixed with and/or be soluble (or suspended) into other types of liquids, including for example, the non-alcoholic wine beverage.

CBD Isolate

In one exemplary embodiment hereof, the CBD may be processed or otherwise transformed into a water-soluble powdered form to be then added to the non-alcoholic wine beverage. This powered form of CBD may be referred to as CBD isolate. CBD isolate may be a crystalline white powder that may comprise a high percentage CBD, such as 99 percent or higher.

To form the CBD isolate, the extracted CBD oil may undergo a purifying process, such as chromatography (CPC), winterization or any other type of purification process that may filter out the plant material (such as terpenes), chlorophyll and excess waxes. The filtering process may also preferably remove any trace levels of Tetrahydrocannabinol (THC) that may be in the oil. This may ensure that the resulting CBD oil may be non-psychoactive.

In addition, both CBD and CBD-A may be present in the Cannabis plant, with CBD-A being the naturally growing acidic precursor to CBD. In fact, CBD-A may be more abundant than CBD in certain types of Cannabis such as hemp. It may therefore be preferable that the CBD oil undergo an activation process, such as decarboxylation, to convert CBD-A to CBD. The main two catalysts of the decarboxylation process may be heat and time. This process may remove the carboxyl group and release carbon dioxide. The resulting CBD may readily interact with the endocannabinoid system of the body such that it may be fully bioavailable (available to be used within the body).

The CBD oil may then be further distilled and crystalized (e.g., using vacuum ovens). The result may provide a powered form of crystalline CBD that may be referred to as CBD isolate. CBD isolate may be a crystalline white powder that may comprise a high percentage CBD, such as 99 percent or higher. It may be preferable that the CBD crystals are odorless and flavorless.

In one exemplary embodiment hereof, the CBD isolate may be added to the non-alcoholic wine directly.

Full Spectrum CBD Oil

In one exemplary embodiment, the extracted CBD oil may undergo a process to reduce the size of the CBD oil particles/droplets (as well as the size of any other types of droplets that may be included and desired within the oil). In this way, the CBD oil particles may be small enough to suspend within non-oil based liquids such as water and/or the non-alcoholic wine beverage. The result may be referred to as an emulsion (e.g., an oil-in-water emulsion). The process may include 1) the use of one or more surface-active agents (surfactants) and 2) exposure to high shear forces.

The surfactants may assist in the breakup of the oil droplets by lowering the interfacial tension of the particles. The surfactants may also assist in stabilizing the resulting smaller droplets by adhering to the newly generated interfaces.

The shear forces necessary for the emulsification process may include high shear mixing or stirring, high pressure homogenization, high amplitude ultrasound (sonication), or other methods and/or forces. In any event, it may be preferable that the shear forces overcome the viscous resistance during the formation of the finely dispersed oil droplets.

It may also be preferable that the resulting emulsions include oil particle/droplet sizes that are small enough to be thermodynamically stable. For example, micro-emulsions (measured in micrometers or microns) and/or nano-emulsions (measured in nano-meters) may be preferable.

In one preferred implementation, the oil droplet size of the CBD oil may be reduced to the size of 100 microns or less such that when added to the non-alcoholic wine beverage, the CBD oil droplets may suspend in the wine.

In another preferred implementation, emulsions with mean droplet diameters ranging from less than 10 nm to 1000 nm may be used, with an average droplet size between 100 nm and 500 nm. Other sized droplet diameters may also be used.

In another preferred implementation, kinetic stability may occur when the oil droplet size distribution may be narrow and its mean droplet size may be smaller than about 300 nanometers. At this size, Brownian motion of the droplets may overcome creaming or sedimentation processes that otherwise may cause the eventual coalescence and separation of the oil as a separate layer.

It is understood by a person of ordinary skill in the art, upon reading this specification, that any sized diameter of oil droplets may be used that result in the successful and stable suspension of the oil within the emulsion.

The benefits of using nano-emulsions are multiple. For example, nano-emulsions of CBD oil may improve the bioavailability of the CBD to the human body. The nano-emulsions may include small-sized droplets having greater surface area for providing greater absorption.

In another preferred implementation, the nano-emulsions of CBD oils may include droplet sizes less than 100 nm which may be optically translucent with a high degree of clarity and stability.

In any event, it may be preferable that the CBD oil particle size be small enough within the CBD emulsions such that when the CBD emulsions are added to the non-alcoholic wine beverage the oil particles may not separate from the beverage even after an extended time. It is understood that the non-alcoholic wine beverage may have a shelf life of weeks, months and even years (depending on the type(s) of dealcoholized wine base used and other factors), and that it may be preferable that the CBD oil particles remain suspended in the beverage during the entire life span of the wine (e.g., while at the manufacturing facility, while on the shelf at the store or outlet, while at the residence of the consumer, etc.).

The benefit of using such a microfluidic process (e.g., emulsification) may be that the resulting CBD additive may include not only the CBD cannabinoid, but also other useful cannabinoid extracts as well. This may be referred to as full-spectrum CBD oil.

Full spectrum CBD oil, unlike CBD isolate products, may include a wide range of cannabinoids naturally present in the Cannabis (marijuana or hemp plant) in addition to the CBD. For example, full spectrum CBD oil may contain other cannabinoids such as, but not limited to, Tetrahydrocannabinol (THC), Cannabigerol (CBG), Cannabinol (CBN), Cannabichromene (CBC), Cannabicyclol (CBL), Cannabitriol (CBT), Cannabicitran (CBT), Cannabifuran (CBF) and others.

In addition, some studies have shown that these and other various cannabinoids may work together synergistically in the body causing what is known as the “entourage effect” to provide greater health benefits when fighting pain and inflammation (among other ailments) compared to CBD acting alone (as with the CBD isolate). In this way, the efficacy and health benefits of the CBD oil may be much improved when provided as full spectrum CBD.

Full spectrum CBD may also contain a variety of essential vitamins, minerals, fatty acids, protein, chlorophyll, flavonoids, and fiber. For example, full spectrum CBD oil may contain vitamins A, C, and E along with complex B vitamins such as niacin, riboflavin, and thiamine. Full spectrum CBD oil may also contain beta-carotene (precursor of vitamin A) as well as critical minerals such as zinc, potassium, iron, calcium, phosphorus and other minerals. In addition, up to twenty amino acids (including up to nine essential varieties) may be included in full spectrum CBD oil, as well as fatty acids such as Omega 3 and Omega 6 (known to promote better health, e.g., for the heart).

In addition, full spectrum CBD may include terpenes such as, but not limited to:

1. Limonene: May boost the consumer's mood and provide relief to stress. May also assist with gastric flux issues.

2. Pinene: May improve memory and boost alertness. May also act as an anti-inflammatory.

3. Linalool: May provide a sedative effect and help with depression, insomnia and stress.

4. Caryophyllene: May be an antioxidant as well as an anti-inflammatory agent that may relieve pain and help with insomnia.

5. Humulene: May provide pain relief, have antibacterial properties and help suppress appetite.

6. Others

In one exemplary embodiment hereof, the full spectrum CBD emulsions (micro-emulsions and/or nano-emulsions with reduced oil droplet sizes) may be prepared in any desired concentrations of any Cannabis extracts (e.g., CBD, THC, other cannabinoids, vitamins, minerals, terpenes and other components), and added to the non-alcoholic wine beverage. The emulsions may be fully miscible with the non-alcoholic wine beverage resulting in the appearance of a fully homogeneous mixture. In this way, the emulsified particles of the Cannabis extracts may then remain suspended within the beverage for extended periods of time.

Besides being water-compatible and easily mixed into beverages at essentially any desired concentrations, Cannabis extract nano-emulsions (nano-cannabinoids) may provide exceptionally high bioavailability and therapeutic effect, and may be absorbed by the body very rapidly and completely. The uptake may begin in the mouth almost immediately upon oral administration. This may result in higher potency and a faster onset of action for lower doses.

In some exemplary embodiments, whether adding CBD isolate or full-spectrum CBD oil to the non-alcoholic wine beverage, the non-alcoholic wine may include 25 mg of CBD per each 8 oz-10 oz serving of the beverage. However, other preferable implementations may also include 6 mg-8 mg, 8 mg-18 mg, 18 mg-24 mg, 24 mg-32 mg, 32 mg-60 mg, 60 mg and above, and other amounts of CBD per each 8 oz-10 oz serving of the beverage.

Tetrahydrocannabinol (THC)

In one exemplary embodiment hereof, the non-alcoholic wine beverage may include Tetrahydrocannabinol (THC). THC may be a cannabinoid that may provide psychotropic effects when activated and consumed. The psychotropic effects may cause temporary euphoria and happiness among other desirable effects. Health benefits of THC may include pain relief, relief from nausea and vomiting, brain cell protection (neuroprotection), insomnia relief, PTSD relief, promotion of brain growth, increased appetite, enhanced senses, antibacterial effects, provides antioxidants, anti-inflammatory, acts as a bronchodilator (helps to open airways in the lungs), potential anti-tumor agent, muscle relaxant, acts as an anticonvulsant, and other health benefits.

Similar to CBD, THC may be extracted from the Cannabis plant and processed prior to being added to the non-alcoholic wine beverage. A person of ordinary skill in the art will understand, upon reading this specification, that all of the details described in other sections of this specification relating to CBD and other types of extracts may also directly apply to THC. For example, THC may be extracted from the Cannabis plant using the same or similar techniques as CBD, may be formed into water soluble isolates containing high concentrations of THC, may be formed as full-spectrum THC emulsions containing other cannabinoids, vitamins, mineral, terpenes, etc., may be formed using other methods and/or any combination thereof. In this way, the THC may be added to the non-alcoholic wine beverage in the same or in a similar fashion as the CBD may be added to the beverage.

The full spectrum THC emulsions may include any or all of the cannabinoid extracts as described above with regards to the CBD emulsions, in similar or different concentrations and/or proportions. In this way, the full spectrum THC emulsions may also provide the benefits of the “entourage effect” as the emulsions (micro and nano) may contain a variety of cannabinoids that may synergistically work together within the body once consumed to provide greater health benefits compared to a THC isolate.

In some exemplary embodiments, the non-alcoholic wine may include 1.0 mg-2.5 mg, 2.5 mg-15 mg, 15 mg-30 gm, 30 mg-50 mg, 50 mg-100 mg and/or more of THC per each 8 oz-10 oz serving of the beverage.

In some exemplary embodiments, the non-alcoholic wine may include full-spectrum CBD oil that may include proportions of both CBD and THC. In some preferable implementations, the full-spectrum oil may have CBD:THC ratios of 0:1, 1:2, 1:1, 2:1, 1:0 and other ratios. In some exemplary implementations, the CBD:THC ratios may include 20:1, 15:1, 10:1, 5:1, 3:1 and other ratios.

### Caffeine

In one exemplary embodiment hereof, the non-alcoholic wine beverage may contain caffeine. Caffeine is known to act as a stimulant to provide energy, focus and a feeling of euphoria. Studies have also shown that the consumption of moderate amounts of caffeine may act to ward off dementia and Alzheimer's disease. Caffeine is also known to be non-addictive.

The caffeine may be provided from any source, including but not limited to:

Yerba Mate: Yerba Mate is an infusion made from the leaves of the tree Ilex paraguariensis, has been shown to be hypocholesterolemic, hepatoprotective, a central nervous system stimulant, diuretic, and a benefit to the cardiovascular system. Yerba Mate may also protect the consumer's DNA from oxidation and in vitro low-density lipoprotein lipoperoxidation and has a high antioxidant capacity (the powder may have a ORAC value of 123,900 (μmol TE/100 g). It has also been suggested for obesity management. Yerba Mate tea may also be associated with the prevention of some types of cancers. The caffeine level may be up to 1.1% by weight (dried).

Guarana: The guarana berry or extract may contain up to 200% to 300% more caffeine compared to coffee beans, and has been shown to improve energy levels, alertness and mood. The caffeine may be extracted from the seeds of the fruit, not the juice. The caffeine level may be up to 5% by weight (dried).

Yaupon holly (cassina): Yaupon holly infusions may contain up to 70% the amount of caffeine compared to coffee beans, and is a great source of antioxidants (dried youpon leaves prior to making the tea may have a ORAC value of 98,700 (μmol TE/100 g). The caffeine level may be equal to 0.65% to 0.85% by weight of the dried leaves.

Guayusa: Guayusa may contain a high level of antioxidants from chlorogenic acid and quercetin (an excellent anti-inflammatory), in addition to a high lutein content. Guayusa may also contain the stimulant theobromine which has been shown to improve mood. The caffeine level may be up to 2.9% by weight (dried).

Other sources of caffeine may include coffee beans, cocoa beans, kola nuts, tea leaves (e.g., black tea, matcha tea, green tea, etc.) and other sources of caffeine.

In one presently preferred implementation, natural caffeine may be added to the non-alcoholic wine beverage in the amount of 40 mg to 80 mg of caffeine per 8 oz to 10 oz serving of the beverage. Other amounts of natural caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, Guarana extract may be added to the non-alcoholic wine beverage in an amount resulting in 40 mg to 80 mg of Guarana caffeine per 8 oz-10 oz serving of the beverage. Other amounts of Guarana caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, Yerba Mate extract may be added to the non-alcoholic wine beverage in an amount resulting in 40 mg to 80 mg of Yerba Mate caffeine per 8 oz-10 oz serving of the beverage. Other amounts of Yerba Mate caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, Yaupon holly extract may be added to the non-alcoholic wine beverage in an amount resulting in 40 mg to 80 mg of Yaupon holly caffeine per 8 oz-10 oz serving of the beverage. Other amounts of Yaupon holly caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, Guayusa extract may be added to the non-alcoholic wine beverage in an amount resulting in 40 mg to 80 mg of Guayusa caffeine per 8 oz-10 oz serving of the beverage. Other amounts of Guayusa caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, coffee bean extract may be added to the non-alcoholic wine beverage in an amount resulting in 40 mg to 80 mg of coffee bean caffeine per 8 oz-10 oz serving of the beverage. Other amounts of coffee bean caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, cocoa bean extract may be added to the non-alcoholic wine beverage in an amount resulting in 40 mg to 80 mg of cocoa bean caffeine per 8 oz-10 oz serving of the beverage. Other amounts of cocoa bean caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, kola nut extract may be added to the non-alcoholic wine beverage in an amount resulting in 40 mg to 80 mg of kola nut caffeine per 8 oz-10 oz serving of the beverage. Other amounts of kola nut caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

In one presently preferred implementation, tea leave (e.g., black tea, matcha tea, green tea, etc.) extract may be added to the non-alcoholic wine beverage in an amount resulting in 40 mg to 80 mg of tea leave caffeine per 8 oz-10 oz serving of the beverage. Other amounts of tea leave caffeine may also be used such as 0-10 mg, 10 mg-20 mg, 20 mg-30 mg, 30 mg-40 mg, 80 mg-100 mg, and 100 mg and more, for each 8 oz-10 oz serving of the beverage.

### Sweeteners

Natural sweeteners may also be added to the non-alcoholic beverage to generally flavor the beverage and/or to mask out any undesirable flavors that may be caused by any of the ingredients described above.

In one exemplary embodiment hereof, clarified pineapple juice may be added in the amount of 4%-15% of the overall beverage. Other percentages may also be used.

In one exemplary embodiment hereof, natural sweeteners and/or artificial sweeteners (e.g., monk fruit and/or erythritol) may be added in any amount of the overall beverage.

In one exemplary embodiment hereof, baobab fruit (e.g., baobab juice), may be added in the amount of 1%-15% of the overall beverage. Other percentages may also be used. The baobab fruit may be fermented and/or in powered form.

In one exemplary embodiment hereof, juices may be added to the beverage, including without limitation, raspberry juice, blackcurrant juice, lemon juice, citrus juice (e.g., orange/tangerine, lemon, lime and other citrus juices), blackberry juice, pear juice, peach juice, watermelon juice, apricot juice and other types of juices. If sugar is also added to the beverage, the juices may be added in the amounts of 9%-15%. If sugar is not added to the beverage, the juices may be added in the amounts of 30%-50%. Note that these percentages are meant for demonstration purposes and other percentages may also be used as necessary.

In one exemplary embodiment hereof, acids that may be found in wines may also be added to the non-alcoholic wine beverage. For example, in some preferred implementations, tartaric, malic, citric acids, butyric, lactic and succinic acids, acetic acid, ascorbic, sorbic and sulfurous acids, as well as other types of acids.

Example quantities of possible ingredients that may make up the non-alcoholic wine beverage are shown in Table 1:

TABLE 1

| INGREDIENT | QUANTITY |
|---|---|
| Dealcoholized Wine (Cabernet, Sauvignon | 40%-60% |
| Blanc, Chardonnay, Merlot or other(s)) | |
| Sugar (if used) | 3%-6% |
| Juice Blend (if sugar is added) | 9%-15% |
| Juice Blend (if no sugar is added) | 30%-50% |
| Acid Blend (Citric, Malic, Tartaric | 0.05%-0.10% |
| or other(s)) | |
| Flavors | 0.10%-0.50% |
| Stabilizers | 0.10%-0.30% |
| CBD (if used) | 15 ppm-75 ppm |
| THC (if used) | 15 ppm-75 ppm |
| Caffeine (if used) | 0.01%-0.02% |
| Water | As needed |

It is understood by a person of ordinary skill in the art, upon reading this specification, that the ingredients shown in Table 1, and the quantity (amount) of each ingredient shown, are shown for demonstration purposes and that the non-alcoholic beverage may include some, all or none of the shown ingredients. It is also understood that the non-alcoholic beverage may include other ingredients in addition to those shown. It is also understood that the non-alcoholic beverage may include different amounts of some or all of the ingredients than those amounts shown. For example, some proportions of some ingredients may depend on the proportions of other ingredients (e.g., the quantities of the juice blend(s) may depend on the quantity of the sugar) such that the quantity ranges of some of the ingredients shown may require adjustment depending on the quantities of other ingredients. It is also understood that the scope of the non-alcoholic beverage is not limited in any way by the ingredients it may contain and/or the amounts of each ingredient.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The present invention also covers the exact terms, features, values and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

What is claimed is:

1. A non-alcoholic wine beverage comprising:
a dealcoholized wine base; and
full spectrum cannabidiol (CBD) oil and/or full spectrum tetrahydrocannabinol (THC) oil.

2.-4. (canceled)

5. The non-alcoholic wine beverage of claim 1 wherein the full-spectrum CBD oil includes a nano-emulsion of CBD oil.

6. The non-alcoholic wine beverage of claim 5 wherein the nano-emulsion of CBD oil includes droplets of CBD oil with an average droplet size between 100 nm and 500 nm.

7.-9. (canceled)

10. The non-alcoholic wine beverage of claim 1 wherein the full-spectrum THC oil includes a nano-emulsion of THC oil.

11. The non-alcoholic wine beverage of claim 10 wherein the nano-emulsion of THC oil includes droplets of THC oil with an average droplet size between 100 nm and 500 nm.

12. The non-alcoholic wine beverage of claim 1 wherein the proportion of CBD to THC is chosen from the group: 0:1, 1:2, 1:1, 2:1, 1:0.

13. The non-alcoholic wine beverage of claim 1 wherein the non-alcoholic wine beverage includes 10 mg-40 mg of CBD per each 8 oz-10 oz serving of the beverage.

14. The non-alcoholic wine beverage of claim 1 wherein the non-alcoholic wine beverage includes 2.5 mg-15 mg of THC per each 8 oz-10 oz serving of the beverage.

15. A method for producing a non-alcoholic wine beverage, the method comprising:
(A) providing a wine base;
(B) dealcoholizing the wine base to create a non-alcoholic wine base;
(C) providing full spectrum cannabidiol (CBD) oil and/or full spectrum tetrahydrocannabinol (THC) oil; and
(D) adding the full spectrum cannabidiol (CBD) oil and/or the full spectrum tetrahydrocannabinol (THC) oil to the non-alcoholic wine base of (B).

16.-20. (canceled)
